(19) United States
(12) Patent Application Publication
(10) Pub. No.: US 2007/0149980 A1
(43) Pub. Date: Jun. 28, 2007

(54) Surgical instruments for use in the implantation of tissue repair kit

(75) Inventors: Bahaa Botros Seedhom, Leeds (GB); Jonathan Charles Lorrison, Leeds (GB); Julian Bryant, Leeds (GB); David Beevers, Leeds (GB)

Correspondence Address: KIRTON AND MCCONKIE, 60 EAST SOUTH TEMPLE,, SUITE 1800, SALT LAKE CITY, UT, 84111, US

(73) Assignee: Xiros PLC, Leeds (GB)

(21) Appl. No.: 10/577,887

(22) PCT Filed: Oct. 27, 2004
(86) PCT No.: PCT/GB04/04537

§ 371 Date: Nov. 22, 2006

(30) Foreign Application Priority Data
Oct. 28, 2003 (GB) 0325277.2

(51) Int. Cl. A61F 2/00

(52) U.S. Cl. 606/99

(57) ABSTRACT

A set of surgical instruments for use in the implantation of a tissue repair kit after preparation of a bone site having damaged tissue, in which damaged tissue is first removed and an annular groove is formed around the defect site prior to application of the repair kit comprising a bio-compatible pad to fit on the prepared bone site, and an overlying cover sheet to fit in the groove. The set of surgical instruments includes: an introducer cylinder having a hollow driving head at one end for introducing an outer portion of the cover sheet into the groove; a plunger relatively slidable within the cylinder and having a delivery end engageable with the cover sheet so as to move a main portion of the cover sheet towards the bone site with the outer portion of the cover sheet trailing behind the main portion, and a pad-receiving recess defined between the delivery end of the plunger and the inner wall of the cylinder so that a pad can be introduced into the recess to overlie the main portion of the cover sheet. A bone site preparation device removes damaged tissue from the bone site and forms the annular groove around the site prior to use of the introducer cylinder and plunger.

This invention relates generally to surgical instruments for use in preparing a bone site with damaged tissue, and for implantation of a tissue repair kit on the prepared bone site, and which includes bone sites on animals and humans.

The invention has been developed primarily, though not exclusively, in connection with the repair of damaged cartilage and the repair of cartilage defects in synovial human or animal joints, and in particular to provide further improvement in the art over the disclosure in WO01/39694. However, it should be understood that the invention has wider application to the repair of damaged tissue in other sites in a human or animal body.

According to the invention there is provided an introducer tool for implanting a repair kit on a prepared bone site from which damaged tissue has been removed, and around which a groove into the bone has been formed, said kit comprising a bio-compatible pad to fit on the prepared bone site, and an overlying cover sheet to fit in the groove, and in which the tool comprises:
an introducer cylinder having a hollow driving head at one end for introducing an outer portion of the cover sheet into the groove;
a plunger relatively slidable within the cylinder and having a delivery end engageable with the cover sheet so as to move a main portion of the cover sheet towards the bone site with the outer portion of the cover sheet trailing behind the main portion; and
a pad-receiving recess defined between the delivery end of the plunger and the inner wall of the cylinder when the cylinder and the plunger are relatively adjusted to a pad-implantation position, so that a pad can be introduced into the recess so as to overlie the main portion of the cover sheet:
in which the internal arrangement of the cylinder and the plunger is such that relative withdrawal of the driving head of the cylinder, followed by relative advancing movement, allows the driving head to engage the trailing portion of the cover sheet and to introduce the trailing portion into the groove while the pad engages the bone site and thereby to anchor the pad in position.

By way of background to the invention, there will firstly be described a tissue repair kit, and method of implantation, which are disclosed in more detail in International Publication number WO01/39694, to which reference is drawn.

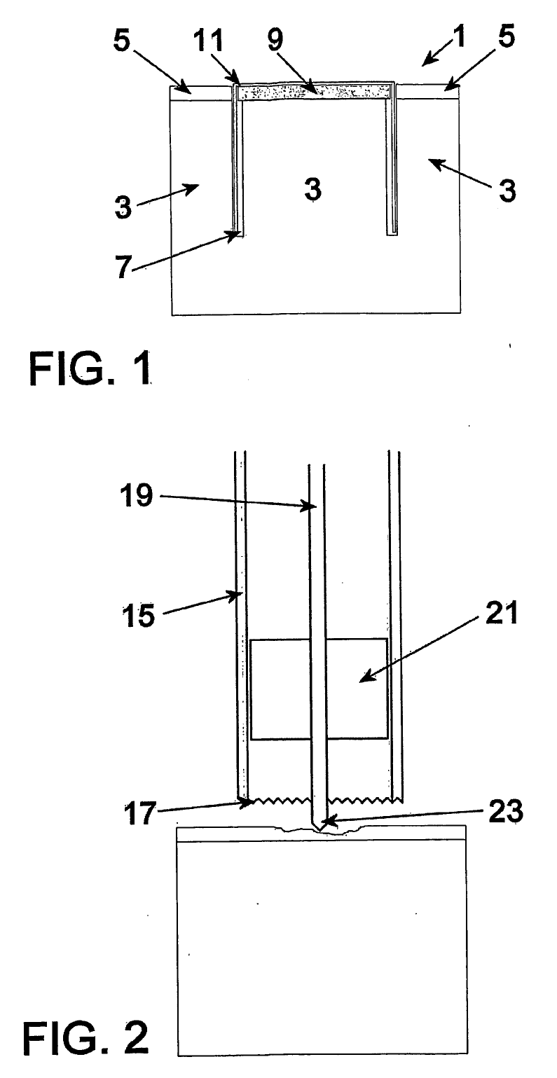

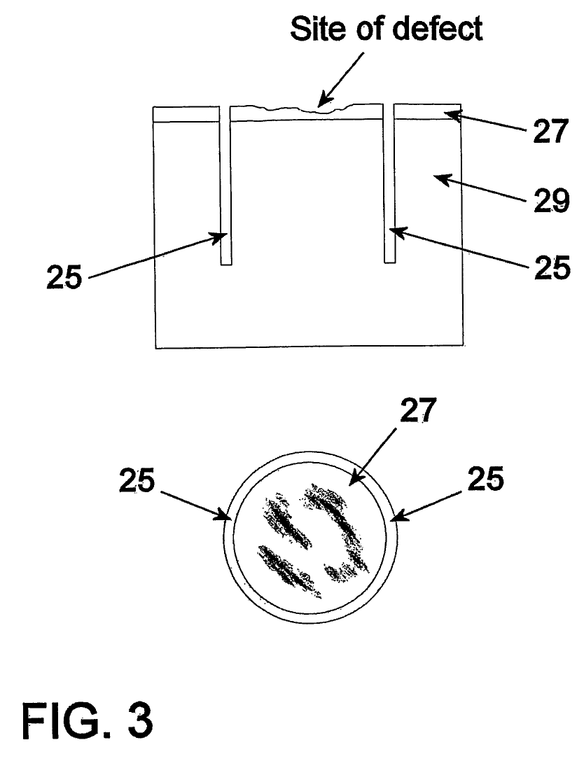

FIGS. 1 to 3 of WO01/39694 illustrate the preparation of a bone site having damaged issue, and the implantation of a repair kit on the prepared site.

Referring therefore to FIGS. 1 to 3 of the accompanying drawings, there is illustrated part of a knee joint 1 including bone 3 overlaid with cartilage 5. An annular space or groove 7 is formed which extends through the cartilage and into the bone, terminating within the bone, at a level that is a multiple of cartilage depth, for example, 4 or 5 times the depth of the cartilage.

Removal of the damaged cartilage from the area of bone defined by the groove 7 results in a space into which is located a small piece or pad of bio-compatible material 9. Pad 9 is shaped and dimensioned to occupy substantially the entire space previously occupied by cartilage, and the depth of pad 9 corresponds approximately to that of the surrounding cartilage 5.

FIG. 2 shows use of a reamer 15 having a toothed edge 17 at one end, and provided with a thin steel rod 19 and near one end there is located a cylinder 21 of external diameter such as to be a snug fit within reamer 15. Adjacent cylinder 21, steel rod 19 has a pointed end 23 enabling rod 19 and its associated cylinder 21, to act as a guide for the reamer 15. In use, the pointed end 23 is located at the centre of the site which includes the damaged cartilage tissue. Light pressure is applied to the steel rod. Reamer 15, located around steel rod 19 and cylinder 21, while being rotated, is then subjected to relatively heavy pressure to cut an annular groove which extends through the cartilage and into the bone, as illustrated in FIG. 1.

Once the material (9) has been positioned at the site from which damaged tissue has been removed, a piece of thin netting/mesh or tissue (11) (forming a cover layer) is then located in the position illustrated in FIG. 1. Mesh 11 extends over the pad (9) and into the annular groove (7) into which it is a push-fit.

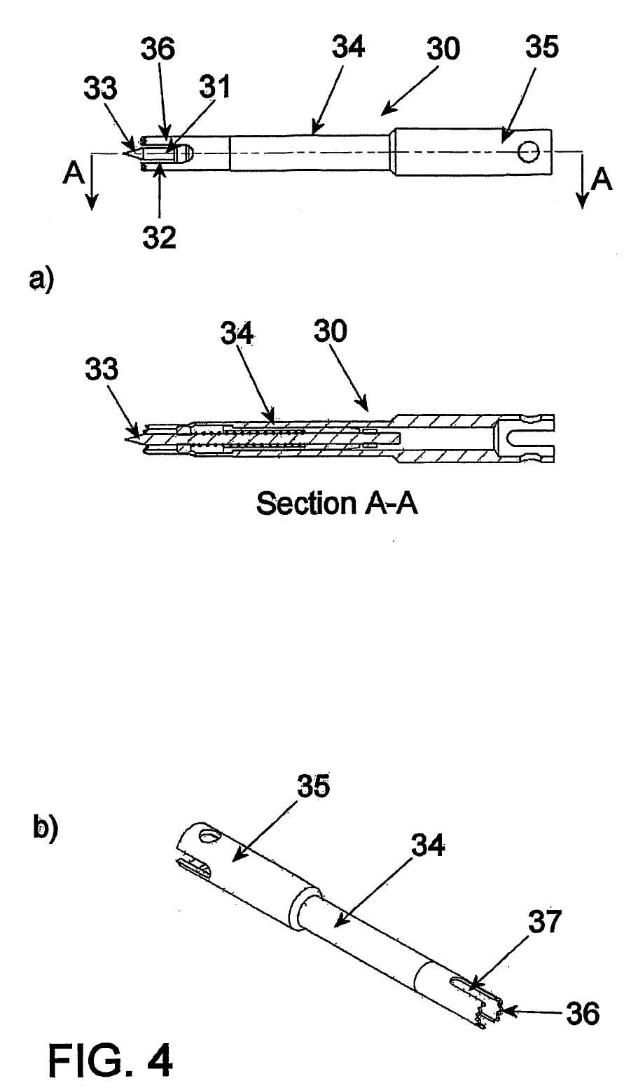

Preferred embodiments of introducer tool and reamer tool according to the invention will now be described in more detail with reference to FIGS. 4 to 10 of the accompanying drawings, in which:

FIG. 4a is a sectional view of an assembled reamer and spring loaded centraliser for use in removing damaged tissue from a bone site;

FIG. 4b is a perspective view of the reamer;

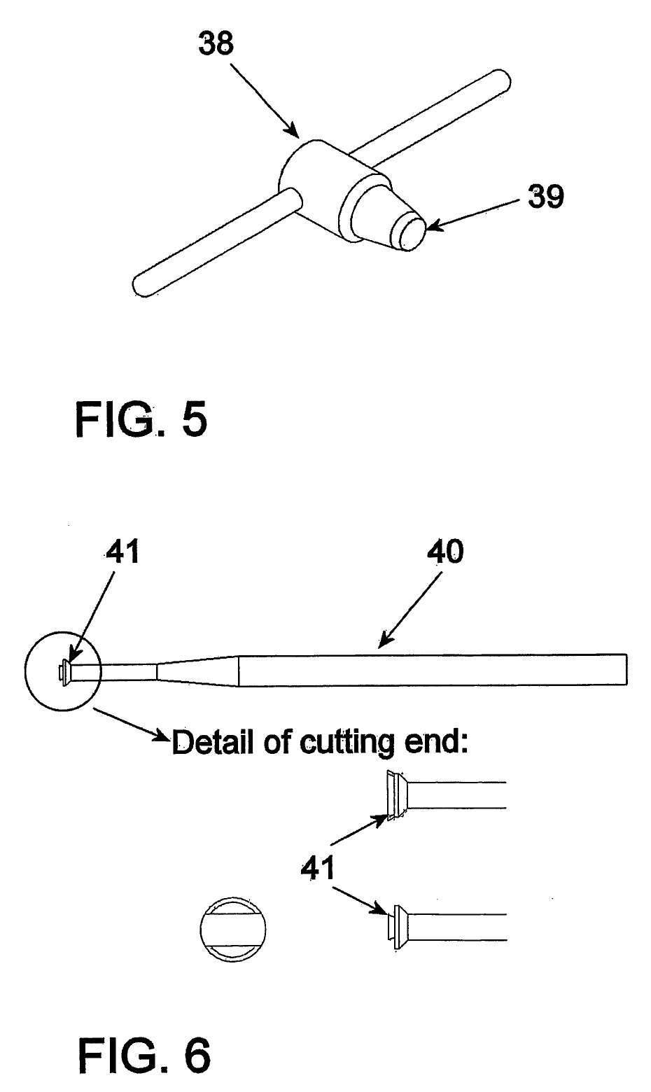

FIG. 5 is a perspective view of a cartilage cutter for use with the reamer;

FIG. 6 is an exploded view of the operating (cutting end) of a residual cartilage removal tool;

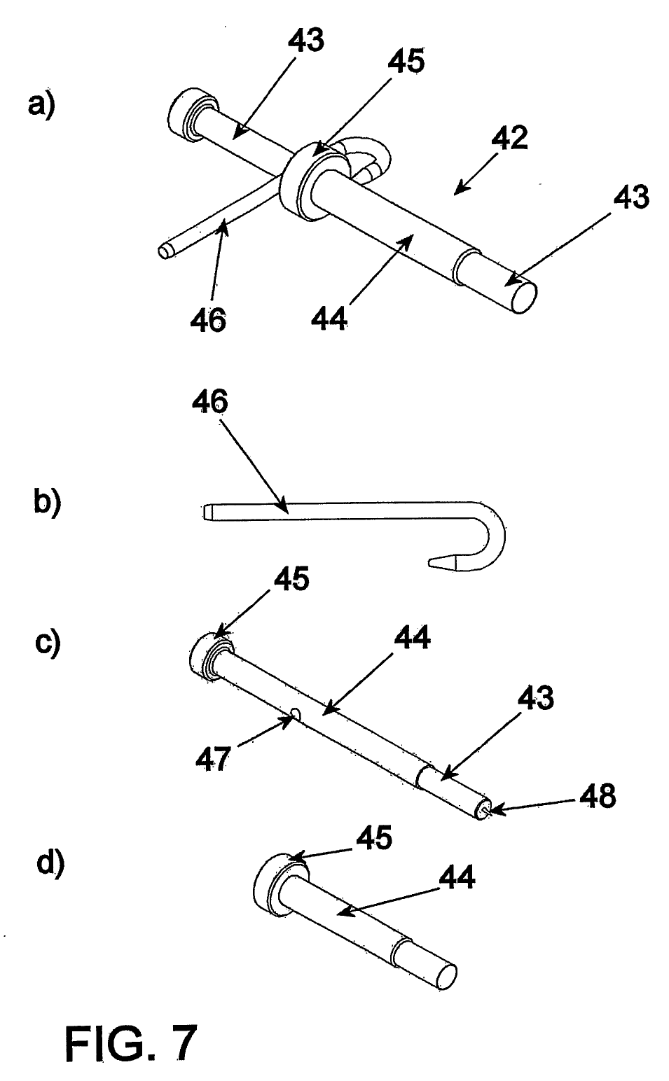

FIG. 7a is a perspective view of an assembly of an introducer/delivery instrument for implanting the repair kit comprising pad and retaining cover sheet;

FIGS. 7b, c and d are illustrations of the component parts of the tools shown in FIG. 7a;

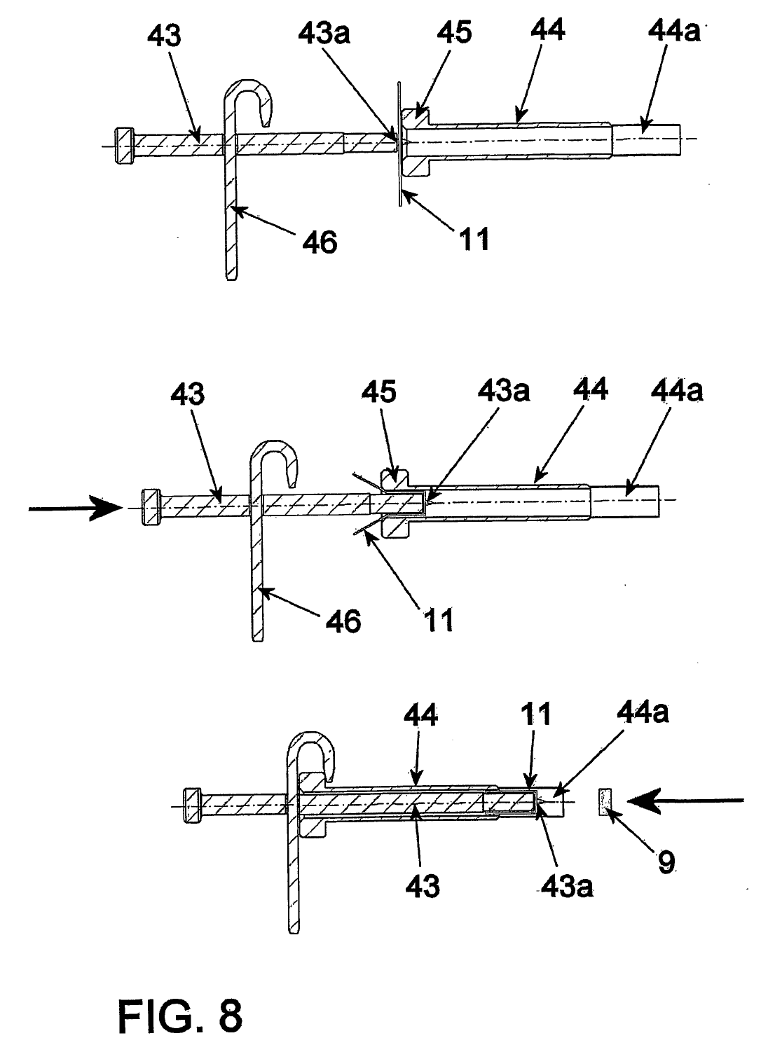

FIG. 8 shows successive views of loading the repair kit into the delivery device/introducer tool;

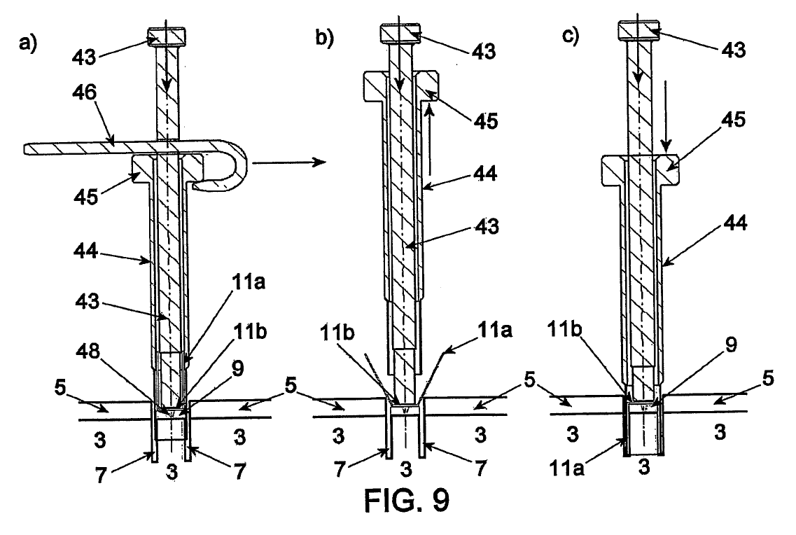

FIGS. 9a, b, c shows successive stages in the implantation of the repair kit on the prepared bone site;

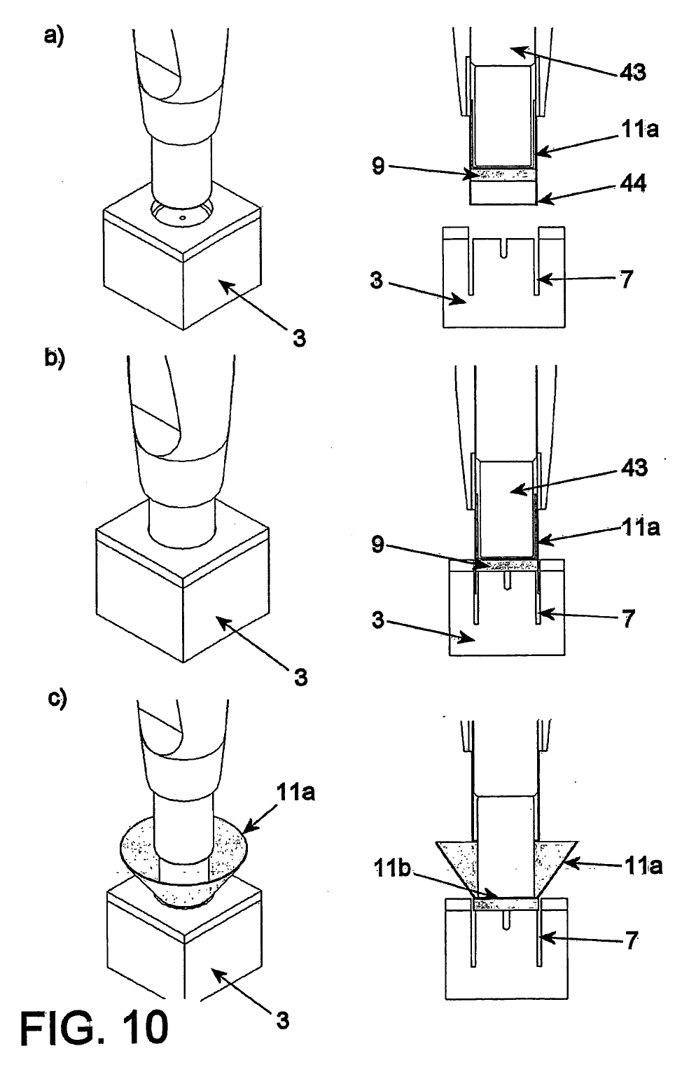

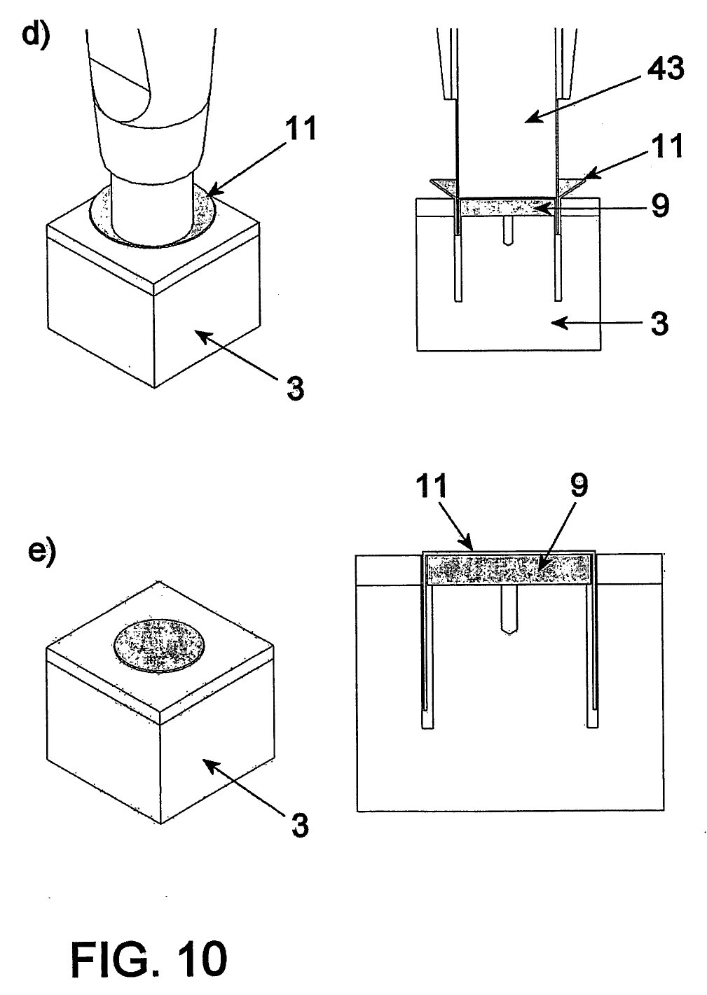

FIGS. 10a to e illustrate in perspective and section the stages of implantation;

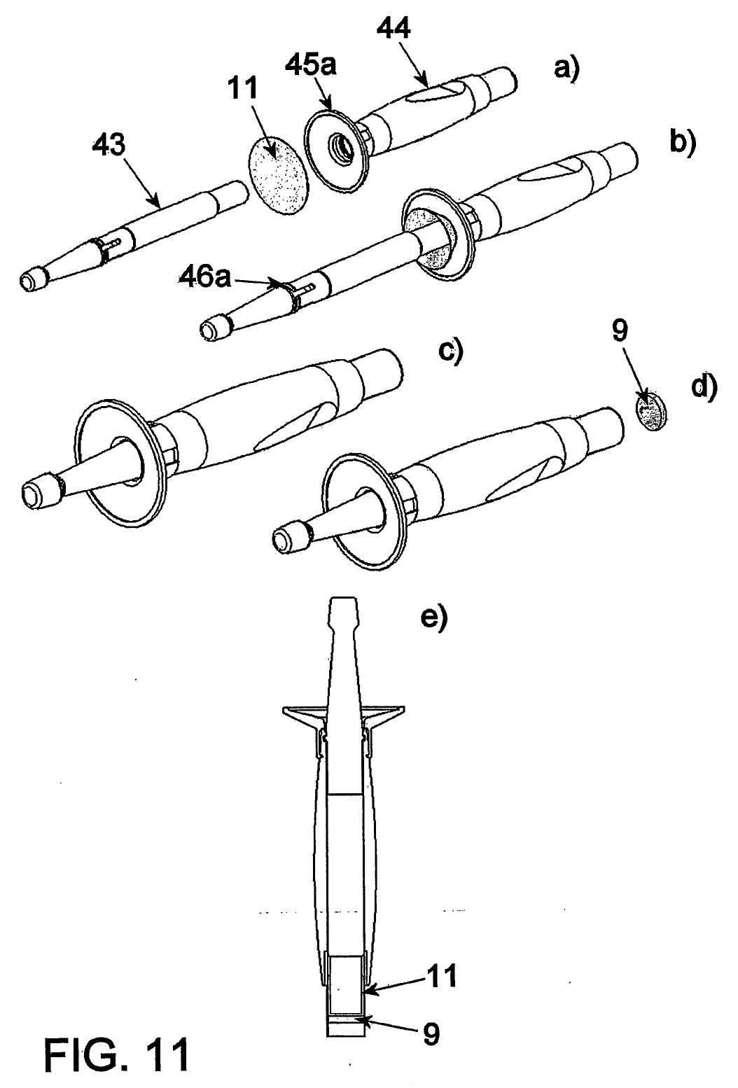

FIGS. 11a to e illustrate in perspective the assembly of the components of the introducer tool, and

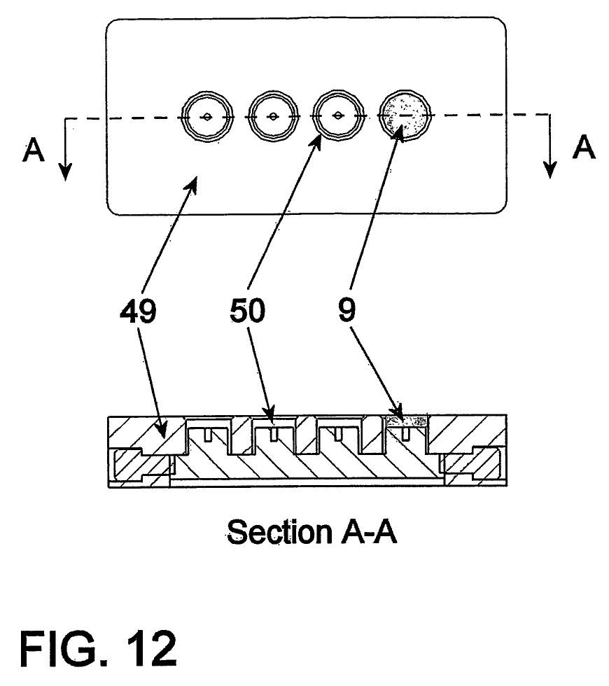

FIG. 12 illustrates the loading of a number of bio-compatible pads in a delivery instrument.

Instruments for Preparing the Repair Site Prior to Implanting the Device (FIGS. 4 and 5)

This is an assembly 30 consisting of stationary and rotating concentric components. The first is a centralising device 31 with a cylindrical section 32 that has at one of its ends a sharp point 33 that pierces the bone and defines the centre of the circle delineating the defect site. This centralising device is contained within a reaming device 34 that is used to make the annular groove 7 surrounding the repair site, said reamer having a wall thickness similar to the annular groove to be generated in the bone at the repair site. At one end 35 the reamer is adapted to connect to a power drill, and at the other end 36 has sharp teeth that remove bone debris as it penetrates the bone to a depth according to a mark on the body of the reamer. At least one channel 37 is cut at the end 36 of the reamer containing the teeth to allow the debris to escape away from the groove, such channels being some 10-15 mm long and which may be either parallel or inclined to the long axis of the reamer. The centraliser 31 is biased axially outwardly of the reamer 34 by being spring loaded within the reamer 34, and the spring-loaded centraliser and reamer are supplied as separate entities to be assembled before use, of preferably as an assembly comprising both and ready for use. The advantage of the spring-loaded centraliser over the one described in the disclosure in WO01/39694 is that the spring-loaded centraliser allows the surgeon to use one hand, rather than both, for simultaneously maintaining the centraliser engaged with the bone under the pressure of the spring while cutting the annular groove with the reamer.

FIG. 4a illustrates the assembled spring-loaded centraliser within the reamer, and FIG. 4b illustrates the reamer alone with the cut channels at its cutting edge.

In addition to the reamer and centraliser, there is supplied a cartilage cutter 38 in the form of a cylindrical component (illustrated in FIG. 5) that is used in conjunction with the reamer 34 and spring-loaded centraliser assembly 31 and when used it surrounds the reamer closely. It has a sharp cutting circular edge 39 at its end near the cartilage surface and such cutter is used to effect a circular cut in the cartilage to its full depth. This is done by pressing the cutter 38 against the cartilage surface while rotating the cutter 38 around the body of the reamer 34. The cartilage cutting is performed after engaging the centraliser into the bone at the repair site, but before making the groove 7 into the bone with the reamer 34. Thus while the centraliser 31 is in place, the cartilage is first cut with the cutter 38 which is then held with its cutting edge 39 in its final position at the cartilage/bone interface and the reamer 34 is then rotated to make the groove 7 to the required depth. Keeping the cutter 38 at the cartilage bone interface protects the edge of the sound cartilage surrounding the repair site from damage were this to come in contact with the rotating surface of the reamer 34. The advantage of this instrumentation assembly for preparing the annular groove is that the surgeon can use one hand to engage the centraliser and later to operate the drill, while with the other hand he/she can operate the cartilage cutter 38 and maintain it in its position at the cartilage bone interface, while operating the power drill to make the groove in the bone.

Instruments for the Removal of Residual Cartilage in the Repair Site (FIG. 6)

Included also is a guard to protect the sound cartilage edge delineating the repair site while removing the residual cartilage from the site. The guard (not shown) is a single component in the form of a thin cylindrical sleeve which is placed within the annular groove whilst removing the residue of cartilage with a rotating tool 40. The end of tool 41 is machined flat on either side leaving a central portion in the form of a dove tail with two cutting edges as shown in the details of FIG. 6. The advantage of this cutting edge configuration is that it can reach to the edge of cartilage close to the guard within the defect site and also that the tool can be effectively used rotated in one direction or the reverse.

Instruments for Implanting Devices for Cartilage Repair (FIG. 7)

The following firstly describes improved instruments for the process of implanting the device described in the disclosure in WO01/39694 in which one of the configurations used a device for cartilage repair comprising a pad and cover sheet for retaining the pad in the annular groove. The instruments described below comprise a delivery instrument 42 for the implantation of the device through the necessarily small incisions made to access the repair site and to facilitate loading the delivery instrument.

The delivery instrument 42 consists of a flat-ended circular plunger 43 placed and slideable within an outer thin walled introducer cylinder 44 the thickness of the wall of the outer cylinder and its diameter are such that it can push the retaining cover sheet 11 within the annular groove 7. The outer component, namely cylinder 44, has a circular flange 45 at one end that is of a diameter substantially equal to or slightly larger than the diameter of the cover sheet 11. The plunger 43 protrudes beyond this flange by a length that facilitates operation, while its other end 43a stops inside some 5-7 mm distance from the end of the outer component. The plunger may or may not have a thin central pin 48 (see FIG. 7c) protruding from its end contained within the outer thin walled component, and has some means of retaining it in that position. By way of example, a removable abutment may limit the insertion movement of the plunger 43 and may take the form of a horizontal through pin 46 extending through hole 47; this prevents it from sliding further into the outer component 44 when introduced through it via its flanged end. The assembled delivery instrument is shown in FIG. 7a and its constituent components in FIGS. 7b, 7c, and 7d.

The method of using this delivery instrument is as follows (see FIGS. 7 and 8):

The device 42 is disassembled, keeping the through pin 46 attached to the plunger 43. One or two of the retaining cover sheets 11 are placed onto the flange 45, concentrically. The plunger 43 is then pushed onto the cover sheet(s) through the flange hole and is prevented from sliding within the thin walled outer component beyond the limit set by the through pin 46. The cover sheets 11 are thus dragged into the thin walled introduces cylinder 44 and kept furled around the plunger (pierced by the pin 46 in its flat end), when a pin is provided. Pads 9 of the required number for the repair site are then placed within a hollow driving head 44a of the cylinder 44 of the outer component 44 and pushed to lie flat against the flat end of the plunger 43. If the latter has a central pin the latter will pierce the pad(s) centrally. The method of loading the delivery instrument described above is shown in FIG. 8.

To implant the device into an already prepared site (with the annular groove made and the residual cartilage removed) having loaded the device (i.e. pads 9 and cover sheets 11) into the delivery instrument as described above, the delivery instrument is introduced into the annular groove and pushed into it to the limit set by the space between the pads within and the edge of the instrument. The outer component is kept at that position within the annular groove, maintaining pressure on the free end of the plunger so as to engage the pin in the flat end of the plunger into the bone at the centre of the repair site. The horizontal retaining pin 46 is withdrawn from the plunger 43 and the outer component 44 is slid around the plunger 43 away from the annular groove until it releases the curled cover sheets captive within it, allowing the cover sheets to unfurl. While maintaining pressure on the plunger end, thus keeping the pads located centrally within the repair site, the cover sheets are pushed into the annular groove with the outer cylindrical component. The pin in the flat-ended plunger would maintain the central location of the pads within the repair site while pushing the cover sheets with the outer thin walled component. The successive steps for implantation of the repair implant are illustrated in FIGS. 9a, b, and c.

FIGS. 10 and 11 represent preferable and practical embodiment of the introducer.

Referring to FIGS. 9a to c, and also corresponding FIGS. 10a to e and 11a to e, there will now be described in more detail the method of use of the introducer tool. The introducer cylinder 44 has a hollow driving head 44a at one end for introducing an outer portion 11a of the cover sheet into the groove 7. The plunger 43, which is relatively slidable within the cylinder 44, has a delivery end 43a engageable with the cover sheet so as to move a main portion 11b of the cover sheet towards the bone site with the outer portion 11a of the cover sheet trailing behind the main portion 11b.

A pad-receiving recess is defined between the delivery end 43a of the plunger 43 and the inner wall of the cylinder 44, when the cylinder 44 and the plunger 43 are relatively adjusted to a pad-implantation position, so that a pad 9 can be introduced into the recess so as to overlie the main portion 11b of the cover sheet 11.

The internal arrangement of the cylinder 44 and the plunger 43 is such that relative withdrawal of the driving head 44a of the cylinder 44, followed by relative advancing movement, allows the driving head 44a to engage the trailing portion 11a of the cover sheet 11 and to introduce the trailing portion 11a into the groove 7 while the pad engages the bone site and thereby to anchor the pad in position.

The main portion 11b of the cover sheet 11 is pressed downwardly onto the pad 9 by the delivery end 43a of the plunger, while the driving head 44a of the introducer cylinder 44 introduces the trailing portion 11a into the groove 7.

In this embodiment of the introducer tool the flange 46a is removable from the outer cylinder, and the plunger has an integral ring that engages in a recess within the outer cylinder of the introducer thus limiting the movement of the plunger within the cylinder.

Referring to FIGS. 10a and 12, another component, which facilitates the loading of pads in the delivery instrument, is a rectangular block 3 and 49, which have one or a number of wells 50 prepared to the same configuration and dimensions of a repair site. The block 49 can be supplied with the wells 50 already charged with one or more pads 9 stacked in the different wells, such pads being picked up by the delivery instrument as the latter is introduced into the annular groove within any well. The pads would simply adhere within the inside of the delivery instrument. The block and method of using it are shown in FIG. 10. The upper surface of a well may have a central hole into which the pin in the flat ended plunger of the delivery instrument may pass, piercing a pad centrally as it is picked from a well within the said block.

Accordingly, preferred embodiments of reamer tool and introducer tool according to the invention, as described above with reference to FIGS. 4 to 10, are improved tools, giving easier preparation of a bone site having damaged tissue, and subsequent implantation of a tissue repair kit, formed of a bio-compatible pad and overlying cover sheet.

In particular, the introducer tool serves to implant a repair kit on a prepared bone site (see FIGS. 1 to 3) from which damaged tissue has been removed, and about which a groove 7 into the bone 3 has been formed, in which the kit comprises a bio-compatible pad 9 to fit on the prepared bone site, and an overlying cover sheet 11 to fit in the groove, to anchor the pad in position.

The tool comprises an introducer cylinder 44 having a hollow circular driving head at one end for introducing an outer portion of the cover sheet into the groove.

A plunger 43 is relatively slidable within the cylinder 44 and has a delivery end onto which the cover sheet can be placed, prior to entry of the plunger into the cylinder, and so as to move a main portion of the cover sheet towards the bone site with the outer portion of the cover sheet trailing behind the main portion.

A pad-receiving recess is defined between the delivery end of the plunger, and the inner wall of the cylinder when the cylinder and the plunger are relatively adjusted to a pad-implantation position, so that a pad can be introduced into the recess so as to overlie the main portion of the cover sheet.

Subsequently, relative withdrawal of the driving head of the cylinder, followed by relative advancing movement, allows the driving head to engage the trailing portion of the cover sheet and to introduce the trailing portion into the groove while the pad engages the bone site and thereby to anchor the pad in position.

1. An introducer tool for implanting a repair kit on a prepared bone site from which damaged tissue has been removed, and around which a groove into the bone has been formed, said kit comprising a bio-compatible pad to fit on the prepared bone site, and an overlying cover sheet to fit in the groove, and in which the tool comprises:
an introducer cylinder having a hollow driving head at one end for introducing an outer portion of the cover sheet into the groove;
a plunger relatively slidable within the cylinder and having a delivery end engageable with the cover sheet so as to move a main portion of the cover sheet towards the bone site with the outer portion of the cover sheet trailing behind the main portion; and
a pad-receiving recess defined between the delivery end of the plunger and the inner wall of the cylinder when the cylinder and the plunger are relatively adjusted to a pad-implantation position, so that a pad can be introduced into the recess so as to overlie the main portion of the cover sheet:
in which the internal arrangement of the cylinder and the plunger is such that relative withdrawal of the driving head of the cylinder, followed by relative advancing movement, allows the driving head to engage the trailing portion of the cover sheet and to introduce the trailing portion into the groove while the pad engages the bone site and thereby to anchor the pad in position.

2. An introducer tool according to claim 1, including a flange integrally, or removably at an entry end of the cylinder against which the cover sheet can be located, prior to insertion of the plunger into the cylinder (44).

3. An introducer tool according to claim 1, including means for limiting the movement of the plunger within the cylinder, and thereby define the pad-receiving recess between the end of the plunger and the surrounding wall of the cylinder.

4. A introducer tool according to claim 3, including a flange integrally, or removably at an entry end of the cylinder against which the cover sheet can be located, prior to insertion of the plunger into the cylinder, in which the removable abutment comprises a retaining pin receivable by a transverse hole in the plunger, and engageable with said flange in order to limit the movement of the plunger within the cylinder.

5. An introducer tool according to claim 1 4, including a pad-loading block having a number of cells each arranged to receive at least one pad, said block being co-operable with the plunger to allow the plunger to be loaded with at least one pad.

6. An introducer tool according to claim 5, in which the plunger has a projecting pin to facilitate removal of a pad from the block, and to attach the pad to the plunger.

7. A bone site preparation device for removing damaged tissue from a bone site and for forming an annular groove around the site, said device comprising:
a reamer for forming the groove;
a centralising device (31) housed within the reamer and having a pointed end for piercing the bone and defining a centre of a circle delineating a defect site of the bone; and
means biassing the pointed end axially outwardly of the reamer to engage with the bone whereby a surgeon may operate the device via one hand by simultaneously maintaining the centralising device engaged with the bone while cutting the annular groove with the reamer.

8. A device according to claim 7, in which the reamer has cutting teeth at one end, and a debris channel formed in said one end to allow bone fragments formed by the teeth to escape while the annular groove is being formed.

9. A device according to claim 7, including a cylindrical cutter for removing damaged tissue and which can inter-fit with the reamer to be rotatable relative to the reamer.

10. A device according to claim 7, in which the end of the reamer remote from the centralising device is adapted to be coupled to a power tool.

11. A device according to claim 7, including a cylindrical guard and a cutting tool, rotatable by the guard in which the guard is capable to being received by the groove while the cutting tool removes damaged tissue surrounded by the groove.

12. A method of implanting a repair kit on a prepared bone site from which damaged tissue has been removed, and around which a groove into the bone has been formed, said kit comprising a bio-compatible pad to fit on the prepared bone site, and an overlying cover sheet to fit in the groove, and comprising the following steps:
introducing an outer portion of the cover sheet into the groove using an introducer cylinder having a hollow driving head at one end which engages the cover sheet;
relatively sliding a plunger within the introducer cylinder so that a delivery end of the plunger engages with the cover sheet and moves a main portion of the cover sheet towards the bone site with the outer portion of the cover sheet trailing behind the main portion;
introducing a pad into a pad-receiving recess defined between the delivery end of the plunger and the inner wall of the cylinder when the cylinder and the plunger are relatively adjusted to a pad-implantation position, wherein the pad overlies the main portion of the cover sheet; and
introducing the trailing portion of the cover sheet into the groove by engagement of the driving head of the cylinder with the trailing portion while the pad engages the bone site and is anchored in position.

13. A method according to claim 12, in which relative withdrawal of the driving head of the cylinder is followed by relative advancing movement, which allows the driving head to engage the trailing portion of the cover sheet and to introduce the trailing portion into the groove while the pad engages the bone site and is anchored thereby.
